(19) United States
(12) Patent Application Publication
(10) Pub. No.: US 2003/0235560 A1
(43) Pub. Date: Dec. 25, 2003

(54) Mastitis prevention

(76) Inventor: Richard J. Harrison, Hockessin, DE (US)

Correspondence Address: Richard J. Harrison, 8 Spring Meadow Lane, Hockessin, DE, 19707, US

(21) Appl. No.: 10/420,121

(22) Filed: Apr. 23, 2003

Related U.S. Application Data
(62) Division of application No. 09/854,445, filed on May 14, 2001, now abandoned.
(60) Provisional application No. 60/203,817, filed on May 12, 2000.

(51) Int. Cl. A61K 45/00

(52) U.S. Cl. 424/93.6

(57) ABSTRACT

The present invention is a method for preventing the onset of mastitis in female mammals. The specification discloses the incorporation of one or more lysogenic bacteriophages with specificity to mastitis causing bacteria into a formulation which is applied to a female mammal's udders.

### CROSS-REFERENCE TO RELATED APPLICATIONS

This is a division of application Ser. No. 09/854,445, filed May 14, 2001 by the present inventor and claims the benefits thereof application Ser. No. 09/854,445 claims the benefits of provisional application Serial No. 60/203,817, filed: May 12, 2000

### STATEMENT REGARDING FEDERALLY SPONSORED RESEARCH OR DEVELOPMENT

Not Applicable

### BACKGROUND OF THE INVENTION

Mastitis is an inflammation of the mammary gland. It is generally caused by microorganisms, usually bacteria, that invade the udder, multiply and produce toxins that damage to the mammary gland. This invention relates to the prevention of mastitis in milking mammals, and more specifically to cows and goats. For purposes of this specification the words udders and teats are used interchangeably as they relate to the mammary gland.

“Mastitis and Its Control”, a paper in the National Dairy Database establishes the significant economic losses caused by mastitis in dairy cattle. These losses which include reduced production, discarded milk, early cow replacement costs, reduced cow sale value, drugs, veterinary services and labor are estimated to be $181 per cow or approximately $1.6 billion in the United States dairy herds alone. These are very visible losses to the dairy producer and thus mastitis has been the subject of significant research.

Results of the aforementioned research indicate that over 95% of mastitis cases are caused by bacterial infection of the udders. Much effort has been put into remedying the widespread and costly bacterial mastitis infections. Various improved methods for pre-milking treatment of udders and methods for prevention of bovine mastitis have been described (see, e.g. U.S. Pat. No. 4,206,529 to Neumann; U.S. Pat. Nos. 5,124,145 and 5,234,684 to Sordillo, et al.: U.S. Pat. No. 4,253,420 to Hoefelmayr.; and U.S. Pat. No. 5,355,732 to Zighelboim). Others have described improved systems of general applicability for delivery of pre-milking treatment by moist wipes (see, e.g., U.S. Pat. No. 4,775,582 to Abba, et al.; U.S. Pat. No. 5,762,948 to Blackburn, et al. and U.S. Pat. No. 4,853,281 to Win, et al.). Berg, et al., J. Dairy Sci. 68, 457-461(1985): Pankey, et al. Veterinary Clinics of North America 9, 519-530, 1993; McKinnon, et al., J. Dairy Res. 50, 153-162, 1983, Murdough, et al., J. Dairy Sci. 76, 2033-2038, 1993 and Ansari, et al., Am. J. Infect. Control 19, 243-249, 1991 provide further description of the present state of the art and describe the evolution of udder or teat hygiene in terms of various aspects of the commonly applied procedures for pre- and post-milking bacterial control.

The best know method for prevention of mastitis today is the application of pre and post milking teat dips. The primary active ingredients in teat dips and wipes are biocides and disinfectants which are present to kill the bacteria within a very short period of time. In addition, the teat dips may also contain additives such as glycerin, lanolin, and aloe vera, to protect and moisturize the skin, but do not kill potential bacteria causing mastitis. Currently used biocides and disinfectants include, chlorhexidine digluconate, iodine, linear dodecyl benaene sulfononic acid, Lauricidin®, quaternary ammonium, sodium hypochlorite, iodophor and more recently the bacterial killing enzymes nisin and lysostatin.

Although all of these biocides and disinfectants have been demonstrated to kill bacteria and reduce mastitis levels, mastitis losses for U.S. dairy producers are still in the range of one point six billion dollars per year. Moreover, we believed that mastitis causing bacteria are developing a resistance to antibiotics currently used for treatment of infected animals much the same as is true with human bacteria. This invention targets the need to reduce the onset of mastitis cases and thus improve productivity while reducing the requirement for antibiotics and thus the tendency to develop bacterial resistant strains.

### BRIEF SUMMARY OF THE INVENTION

This invention relates to a bacteriophage or a combination of bacteriophages used prophylactically to prevent the onset of mastitis. Bacteriophages are bacterial killing organisms which can be selected for specific bacteria. The bacteriophage or bacteriophages, which may be used alone or in combination with known tip dip formulation compounds, are incorporated into a teat dip formulation which is applied to the udders by dip, spray or wipe to create a protective coating on the udder skin. More specifically, this invention relates to the incorporation of lysing bacteriophages which are specific to bacteria known to cause mastitis into an udder treatment formulation which is applied to the teat skin to prevent mastitis. Application of the udder treatment formulation may be by any method which provides extensive teat coverage such as dipping, spraying or wiping.

This invention also relates to the composition of teat dip formulations which in addition to the bacteriophages may contain moisturizers and conditioners such as glycerin, lanolin, and aloe vera to protect the skin, and biocides and disinfectants such as chlorhexidine digluconate, iodine, linear dodecyl benaene sulfononic acid, Lauricidin®, quaternary ammonium, sodium hypochlorite, iodophor, nisin and lysostatin. Lastly, the formulation may contain inactive cell debris from host bacteria used to amplify the bacteriophage, thus eliminating a costly step from the manufacturing process.

### BREIF DESCRIPTION OF THE SEVERAL VIEWS OF THE DRAWING

Not Applicable

### DETAILED DESCRIPTION OF THE INVENTION

Bacteriophages (“phages” for short) are viruses whose hosts are bacterial cells. Like all viruses, phages are metabolically inert in their extracellular form (the “virion”), and they reproduce by insinuating themselves into the metabolism of the host. The mechanisms by which phage virions infect their host cells—described in more detail below—vary among the different types of phages, but they all result in delivery of the phage genome into the cytoplasm of the bacterial host, where it interacts with the cellular machinery to carry the phage life cycle forward. The result of infection can be, and often is, total devastation for the cell (lytic infection) and replication and perpetuation of the bacteriophage. Bacteriophages and viruses are basically complex organic heteropolymeric compounds which have the capacity for self-replication. The molecular structure, also known as the nucleocapsid can be dissected into a nucleic acid and a protein component.

Bacteriophages were discovered a little over 80 years ago—in 1915 by the Englishman Frederick Twort and independently in 1917 by the French Canadian Felix D'Hérelle It was quickly realized that bacteriophages had the potential to kill the bacteria that cause many infectious diseases in humans, as well as in agriculturally important plants and animals. During early research the viral nature of the bacteriophage was clearly established, the chemical composition of the virions (the extracellular virus particles) was measured and shown to be protein and DNA, new phages infecting a variety of bacterial hosts were isolated, and some rudimentary progress was made in understanding the virus life cycle.

The modern era of bacteriophage research is usually dated from 1938 when the expatriate German physicist, Max Delbrück, began his work on phages at the California Institute of Technology. The astonishing success of bacteriophage research over the 25-30 years prior to about 1970 in revealing the fundamental “secrets of life” can be attributed largely to the fact that phages are so tractable as experimental systems. That is, they are genetically and structurally simple, they have a short life cycle that can be synchronized in a population, and genetic, biochemical, and structural approaches can be applied synergistically. The fact that phages interact intimately with their bacterial hosts means that virtually everything that is learned about phages is also informative about the bacterial cells they infect, and often about even broader biological questions.

Around 1970 the world of biological research began to be transformed by the ‘recombinant DNA revolution’, with which it becomes possible to effectively change a gene from any organism—no matter how complex or how eukaryotic—into a phage gene. Today, practical uses of phages, include genetic tools (cloning vector, integrating plasmids, etc.), epitope display, making ssDNA sequencing templates, reporter phages, and phage therapy.

The idea of using phage therapy as a mastitis treatment is referenced once in the literature in the Ann. Rech. vet. 1990 volume 11 page 421-426 where Catherine Lerondelle reports on her failed attempt at “BACTERIOPHAGE TREATMENT TRIALS ON STAPHYLOCOCCAL UDDER INFECTION IN LACTATING COWS”. In this experiment Lerondelle attempts to inject cows with staphylococcal bacteriophage. The literature contains no other reference to mastitis treatment with bacteriophages.

It should be noted that the purpose of this invention is not to treat mastitis cases as described in the Lerondelle reference, but rather to prevent the occurrence of mastitis by using the phage as a prophylactic through its incorporation into a dip to be used as a prophylactic against mastitis. Unlike like other disinfectants which are believe to have a relatively short period of effectiveness because of their failure to prevent the occurrence of mastitis, properly formulated bacteriophages will provide protection throughout the period between milkings and thus reduce the presence of bacteria causing mastitis. Because phage multiply in the presence of bacteria, they increase their killing power in the presence of bacteria. Thus repeated bacteria challenges are more likely to be terminated using bacteriophages than by disinfectants which lose their strength with repeated challenges and time. Lastly, bacteriophages are very small organism which can be stored and survive in areas on the udders which are most susceptible to bacterial growth and thus when applied to the udder surface in the form of a dip, spray or wipe can lay dormant until activated by the arrival of mastitis causing bacteria.

This invention contemplates the use of one or a cocktail of bacteriophages specifically targeted to bacteria which have been shown to cause mastitis. The American Tissue Culture Collection (ATCC) catalog lists a number of potential bacteriophages which will attach and kill Streptococcus agalactiae and Staphylococcus aureus bacteria (the two major bacterial causes of mastitis). Examples include ATTC #'s 12169-B1, 21597-B2, and 29200-B1 Streptococcus phages and 11987-B1, 15752-B1 and 27696-B1 Staphylococcus phages.

Bacteriophages reproduce through attacking a host bacteria. Although the bacteriophage may be separated from the host bacteria cells and used as a semi or purified product. A further invention disclosed is the manufacturing portion of the bacteriophage(s). In the proposed manufacturing process, the host bacterial culture for the bacteriophage (which may or may not be a strain of the bacteria which causes mastitis) may be incorporated into the final product with little of no purification. (Some filtration may be required to remove large particles if the bacteriophage is to be produced as an aerosol and nozzle plugage might occur.) This will be accomplished through the addition of a small quantity of disinfectants such as those previously mentioned as being incorporated into udder dips should any living bacteria remain to be killed. A disinfectant will be selected such that it does not inhibit the bacteriophages activity, yet is know to have a 100% kill rate for bacteria in culture (it is well know that bacteria in culture are much easier to kill than environmental bacteria). Thus, it is envisioned that one method of manufacture will be to add a disinfectant to the bacteriophage and the mixture be incorporated into the final udder dip or spray. Such a mixture will provide both short and long term bacterial kill power and provide a long term solution to the mastitis problem.

Method of Treatment

Prophylactic treatments for mastitis according to the invention involve the use of a teat dip formulation containing bacteriophage which are know to lyse mastitis causing bacteria. Bacteriophage-containing teat dips provide effective prevention of bovine mastitis in lactating cows when used after every milking. Preferably, the preventative regimen is used for all cows in the herd. In the preferred embodiment, the teat dips comprise about 5×10⁷ colony-forming units (cfu)/ml in an acceptable carrier. In addition, teat dips for use according to the invention may include a mild surfactant. Acceptable carriers are those which provide an environment in which the bacteriophage remain functional and provide a buffered medium and include aqueous buffers or hydrophilic ointment bases. For example non-ionic detergents, fatty acids or other mild surfactants, protein carriers, such as serum albumin or gelatin, powdered cellulose and carmel can be used as a carrier. The teat dip may also advantageously include chelating agents, EDTA, colorants, and humectants, such as glycerol or sorbitol.

Bacteriophage containing teat dips can also be used as a prophylactic treatment for dry cows. In this case, the bacteriophage is formulated into an aqueous polymer based coating which is used to form a thick film barrier over the teat.

### EXAMPLE 1

Protocol to demonstrate the bactericidal activity of Staphylococcus aureus bacteriophage toward Staphylococcus aureus mastitis associated bacteria

1. Host bacterial cells, Staphylococcus aureus (ATCC) 27702, are grown overnight in ATCC medium, 18 Tryplicase soy agar at 37 degrees C. yielding a final concentration generally in the range of 10⁹ cells/ml.

2. Day 2—Staphylococcus aureus bacteriophage 27702-B1 (ATCC) is introduced into the 27702 active growing broth culture and incubate for 24 hours at 37 degrees C.

3. Staphylococcus aureus 27740 (ATCC) bacteria are grown overnight in ATCC medium: 3 Nutrient agar (Difco 0001) at 37 degrees C. yielding a final concentration generally in the range of 10⁹ cells/ml.

4. Day 3—Serially dilute the Staphylococcus aureus 27702-B1 bacteriophage seven times.

5. Prepare 0.5% soft-agar overlay plates of the actively growing 27740 bacteria.

6. Add one drop of the 27702-B1 serial dilution sample to the hardened overlay and incubate overnight. (Three to four dilution's can be placed on each plate).

7. Day 4—Lysis should be visible on the plates and at higher dilutions individual plaques should be countable.

Composition of Soft-Agar Per Liter

Difco casamino acids, 3.0 g; Difco yeast extract, 3.0 g; NaCl, 5.9 g; Na lactate (60% w/v),

3.3 ml; 25% (v/v) glycerol, 4.oml; agar, 15 g; pH adjusted to 7.8.

Composition of Trypticase Soy Agar Per Liter

Bacto Tryptone, 15 g; Bacto Soytone, 5 g; NaCl, 5 g; agar, 15 g; pH adjusted to pH 7.3.

### EXAMPLE 2

Protocol to demonstrate the efficacy of bacteriophage teat-dip compositions in vivo. Protocol A of the National Mastitis Council is used as the basis for testing. In general, teats are cleaned with a 1% iodine solution and dried with a paper towel. Teats are then rinsed with alcohol and allowed to air dry. All four teats per cow are next dipped in a 10⁸ cell/ml suspension of Staphylococcus aureus strain Newbould 305 to cover ½ the teat and allowed to air dry for 30 minutes. Two teats (right fore and left rear) are then dipped in a bacteriophage teat dip formulation (10⁶ phage/ml in 0.85% saline) to cover ⅔ of the teat and allowed to air dry for 30 minutes: the remaining two teats act as non-treated controls. Each teat is first swabbed with a moist cotton swab and then washed with 10 ml of 0.85% sterile saline solution: the wash is collected into a sterile tube. A 0.2 ml sample of the wash, and appropriate dilutions thereof, are plated on blood agar in duplicate and incubated at 37 degrees C. for 24-48 hours, Colony forming units are determined and percent survival of Staphylococcus aureus calculated relative to controls.

### EXAMPLE 3

Preparation and application of a commercial teat dip for lactating bovine. At least one and preferably three or four lysogenic phage stocks per Streptococcus agalactiae, Staphylococcus epidermidis, Staphylococcus aureus, Streptococcus dysgalactiae, Streptococcus bovis and Corynebacteriaum bovis bacteria are grown separately in their host bacteria to a final stock concentrations generally in the range of 10⁹ phage/ml. Any remaining viable host bacteria in each phage stock are killed using the minimum amount of a detergent agent such as glyceryl monolaurate to disrupt the bacterial cell membranes. Each phage stock is then centrifuged to remove cell debris and serially plated to determine phage concentration of each stock. A one liter batch of phage dip is prepared by adding a sufficient quantity of each phage stock to provide a concentration of 5.×10⁷ of each phage in the batch. The phage cocktail is then mixed with 10 ml of aloe and diluted to one liter with buffer. The teat dip is ready for application after mixing. It should be applied to each teat by dip, spray or wipe after each milking for maximum efficacy.

### EXAMPLE 4

Preparation and Application of a Teat Coating for Dry Cows.

At least one and preferably three or four lysogenic phage stocks per Streptococcus agalactiae, Staphylococcus epidermidis, Staphylococcus aureus, Streptococcus dysgalactiae, Streptococcus bovis and Corynebacteriaum bovis Phage are prepared as previously described. Each phage stock is then mixed with an aqueous film forming teat coating to achieve a final concentration for each phage of 5.×10⁷. The film forming barrier coating is then applied to the dry cow teats.

What I claim as my invention is:

1. A method for preventing the onset of mastitis in a female mammal which comprises coating a mammal's udders with a formulation containing one or more bacteriophages specific to bacteria which are know or suspected of causing mastitis.

2. The method of claim 1, wherein said female mammal is by scientific classification a member of the bovidea family.

3. The method of claim 2, wherein said female mammal is a cow, bison, buffalo, oxen, goat or sheep.

4. The method of claim 1, wherein said female mammal is by scientific classification a member of the tayassuidae family.

5. The method of claim 1, wherein an udder coating for lactating female mammals is applied to the udder surface after each milking.

6. The method of claim 1, wherein a barrier udder coating containing one or more phages is applied to the udders of non-lactating female mammals.

7. The method of claim 1, wherein the solution containing one or more bacteriophages may also incorporate additional compounds found in traditional udder dips.

8. The method of claim 7, wherein the additional compounds shall include but not be limited to moisturizers, disinfectants, antimicrobials, aqueous buffers and hydrophilic ointment bases.

9. The method of claim 1, wherein the bacteriophage or bacteriophages are lysogenic to mastitis causing bacteria.

10. The method of claim 9, wherein the bacteriophages incorporated may include but are not exclusively limited to those bacteriophages which lyse Streptococcus agalactiae, Staphylococcus epidermidis, Staphylococcus aureus, Streptococcus dysgalactiae, Streptococcus bovis and Corynebacteriaum bovis.
